(19) United States
(12) Patent Application Publication
(10) Pub. No.: US 2012/0245554 A1
(43) Pub. Date: Sep. 27, 2012

(54) Micro-Infusion System

(75) Inventor: Yasuhiro Kawamura, Tokyo (JP)

(73) Assignee: K & Y Corporation

(21) Appl. No.: 13/424,238

(22) Filed: Mar. 19, 2012

Related U.S. Application Data
(60) Provisional application No. 61/453,909, filed on Mar. 17, 2011.
(60) Provisional application No. 61/566,542, filed on Dec. 2, 2011.
(60) Provisional application No. 61/611,452, filed on Mar. 15, 2012.

(51) Int. Cl. A61M 5/168; A61M 5/142

(52) U.S. Cl. 604/500; 604/151; 604/67

(57) ABSTRACT

Infusion systems according to the present invention provide a medical fluid infusion system operable at a relatively wide range of flow rates while simultaneously maintaining a high degree of accuracy and predictability through employing specific flow path architecture, flow path dimensional ranges, and pump control parameters, such as voltage, frequency, voltage rise time, pump size and quantity, and controlled restriction of the fluid flow path. Automatic recognition of restrictive elements is employed to facilitate the ease of use of different restrictive elements with a single infusion system and improve patient safety.

### RELATED APPLICATIONS

This application claims priority to U.S. Provisional Application Ser. No. 61/611,452 filed Mar. 15, 2012, entitled Infusion System; U.S. Provisional Application Ser. No. 61/566,542 filed Dec. 2, 2011, entitled Infusion Pump; and U.S. Provisional Application Ser. No. 61/453,909 filed Mar. 17, 2011, entitled Infusion Pump, each of which is hereby incorporated herein by reference in its entirety.

### FIELD OF THE INVENTION

The present invention relates to medical infusion systems and related methods and, more particularly, to infusion systems employing a piezoelectric effect for medical and healthcare related applications.

### BACKGROUND OF THE INVENTION

Fluid pumps can be driven based on various design principles including the piezoelectric effect. The piezoelectric effect can be employed to indirectly cause fluid flow, for example a piezoelectric driven motor or actuator can be used to linearly displace a plunger to push fluid from a reservoir or to rotate a rotor in a peristaltic-type pump. For example, U.S. Publication Nos. 2009/0124994 to Roe and 2009/0105650 to Wiegel et al., and U.S. Pat. Nos. 7,592,740 to Roe, and 6,102,678 to Perclat teach the application of such technologies to infusion pumps used in the medical and health care industries.

Alternatively, the piezoelectric effect can be employed to cause fluid flow through the direct manipulation of a fluid chamber or flow path, for example through vibration of an internal surface of a fluid chamber. Such microelectromechanical system, or MEMS, micropumps can be fabricated using known integrated circuit fabrication methods and technologies. For example, using integrated circuit manufacturing fabrication techniques, small channels can be formed on the surface of silicon wafers. By attaching a thin piece of material, such as glass, on the surface of the processed silicon wafer, flow paths and fluid chambers can be formed from the channels and chambers. A layer of piezoelectric material, or a piezoelectric body such as quartz, is then attached to the glass on the side opposite the silicon wafer. When a voltage is applied to the piezoelectric body, a reverse piezoelectric effect, or vibration, is generated by the piezoelectric body and transmitted through the glass to the fluid in the chambers. In turn, a resonance is produced in the fluid in the chambers of the silicon wafer. Through the inclusions of valves and other design features in the fluid flow paths, a net directional flow of fluid through the chambers formed by the silicon wafer and the glass covering can be achieved.

MEMS micropumps have become an established technology in the inkjet printer industry. Technological developments relating to increased definition and ink throughput for piezoelectric micropumps, or MEMS micropumps, for inkjet printers have achieved more precise printing with smaller ink throughputs. For example, it has become possible to control the ink throughput of inkjet printers employing MEMS micropumps at the picoliter level. Furthermore, in order to address the problems associated with uneven printing in inkjet printers due to the vaporization of gas dissolved in the ink, considerable development has also been directed to providing inkjet printers with structures for degassing the ink.

MEMS micropumps employing the piezoelectric effect have also been contemplated for use in small and large-volume infusion pumps, i.e. pump systems that are typically employed to infuse fluids, medications, and nutrients into a patient's circulatory system. For example, with respect to small-volume infusion systems, U.S. Pat. Nos. 3,963,380 to Thomas, Jr. et al.; 4,596,575 to Rosenberg; 4,938,742 to Smits; 4,944,659 to Labbe et al.; 5,984,894 to Poulsen et al.; and 7,601,148 to Keller all describe various micropumps intended for implantation into a patient in order to administer small amounts of pharmaceuticals, such as insulin. Similarly, U.S. Publication No. 2007/0270748 to Dacquay et al. describes a piezoelectric micropump integrated into the tip of a syringe for very low volume delivery of ophthalmic pharmaceuticals to a patient's eye.

In contrast to inkjet printers and small-volume infusion micropumps, typical medical infusion pumps must be operable to provide significantly increased fluid throughput. However, as fluid throughput, or fluid flow rates are increased, the potential for the vaporization of dissolved gas correspondingly increases. The vaporization of dissolved gas within the fluid flow paths of infusion pump systems presents a significant health hazard to patients receiving infusions. While the problems associated with the vaporizations of dissolved gas in inkjet printer micropumps, systems in which fluid throughputs are relatively low, has largely been addressed through the development of degassing technologies, satisfactory solutions have not been presented for high-throughput micropumps, such as infusion pumps, used in the health and medical industry. U.S. Publication No. 2006/0264829 to Donaldson and U.S. Pat. No. 5,205,819 to Ross et al. described large-volume infusion systems employing piezoelectric micropumps; however, neither of these systems provides solutions directed to overcoming the problems associated with vaporization of dissolved gas at high fluid throughputs.

What is needed in the field is a highly accurate infusion pump system that provides a relatively wide range of fluid throughput while reducing or eliminating the risks to patients and increasing medical staff efficiency.

### OBJECTS AND SUMMARY OF THE INVENTION

Infusion systems according to the present invention provide a medical fluid infusion system that achieves a relatively wide range of flow rates while maintaining a high degree of accuracy and predictability. Infusion systems according to the present invention achieve these advances by employing specific flow path architecture, flow path dimensional ranges, and pump control parameters, such as voltage, frequency, voltage rise time, pump size and quantity, and controlled restriction of the fluid flow path for generation of back pressure and controlling such characteristics and parameters relative to one another.

In certain embodiments, infusion systems according to the present invention achieve automatic recognition of restrictive elements, thereby facilitating the ease of use of different restrictive elements with a single infusion system and improving patient safety.

In another embodiment of the present invention, the infusion system is incorporated into a fluid bag thereby streamlining the infusion system for bed-side or mobile usage.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a diagram of an infusion system according to one embodiment of the present invention.

FIG. 2 is a partial cross-sectional view of a pump core according to one embodiment of the present invention.

FIG. 3A is a partial cross-sectional view of a pump core and pump stay according to one embodiment of the present invention.

FIG. 3B is a plan view of a pump stay according to one embodiment of the present invention.

FIGS. 4A and 4B are graphs of a control voltage applied to an infusion system according to one embodiment of the present invention.

FIG. 5 is a graph of a control voltage applied to an infusion system according to one embodiment of the present invention.

FIGS. 6A, 6B, and 6C are graphs of a control voltage applied to an infusion system according to one embodiment of the present invention.

FIG. 7 is a diagram of a portion of an infusion system according to one embodiment of the present invention.

FIG. 8 is a diagram of a portion of an infusion system according to one embodiment of the present invention.

FIG. 9 is a cross-sectional view of a pump according to one embodiment of the present invention.

FIG. 10 is a partial cross-sectional view of a flow restriction according to one embodiment of the present invention.

FIG. 11 is a partial cross-sectional view of a restrictive patient line according to one embodiment of the present invention.

FIG. 12 is a partial cross-sectional view of a flow restriction according to one embodiment of the present invention.

FIG. 13 is a side elevation view of a portion of a patient line according to one embodiment of the present invention.

FIG. 14 is a side elevation view of a portion of an outlet connection according to one embodiment of the present invention.

FIGS. 15A and 15B are partial cross-sectional views of auto-recognition features according to one embodiment of the present invention.

FIG. 16 is a side elevation view of alignment features according to one embodiment of the present invention.

FIG. 17 is a side elevation view of an infusion system incorporating a fluid bag according to one embodiment of the present invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

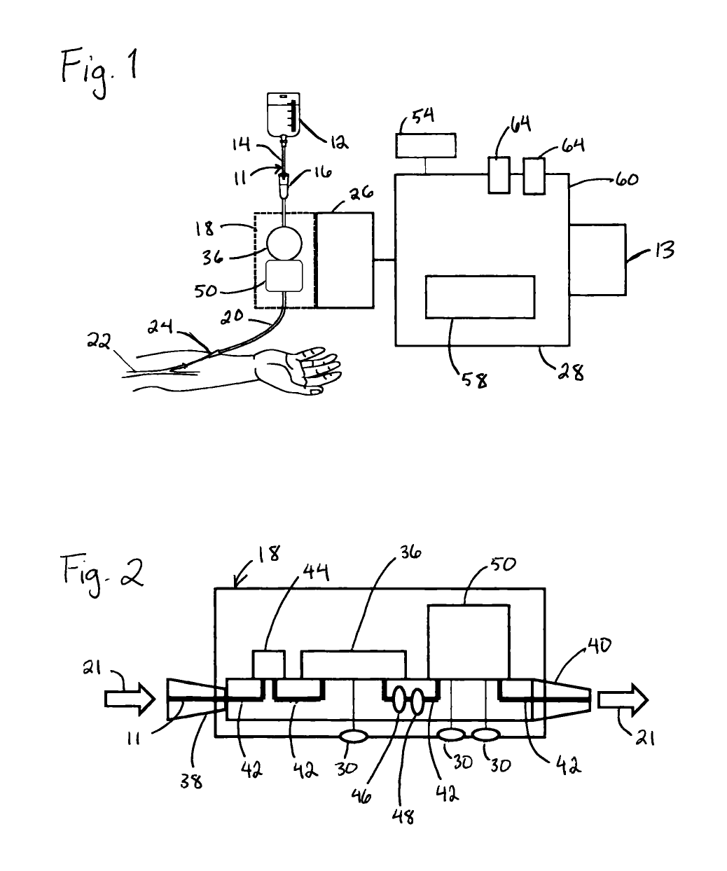

As shown in FIG. 1, a generalized overview of an infusion systems or micro-infusion system 10 according to the present invention includes a patient fluid flow path 11 comprising an administrative set or tube set 14, a pump core 18, a patient line 20, and a connector 24. The administrative set 14 provides fluid communication between an infusion bag 12 and the pump core 18. The administrative set 14 may include a drop cylinder 16 located between the infusion bag 12 and the pump core 18. The patient line 20 provides fluid communication between the pump core 18 and the connector 24. The connector 24 functions as a fluid access point with a patient circulatory system 22. According to one embodiment of the present invention, all of the components of the fluid flow path 11 of the infusion system 10, for example, the administrative set 14, pump core 18, patient line 20, and connector 24 are disposable components of the system 10. The infusion system 10 may also employ a bracket or support structure 13 that functions to secure the system 10 to, for example, a pole or stand.

As shown in FIG. 2, the fluid flow path 11 enters the disposable pump core 18 at the pump core inlet 38 which is in communication with fluid passes 42. For the sake of clarity, arrows 21 indicate the direction of fluid flow through the pump core 18. The fluid passes 42 direct fluid through a filter 44, a pump 36, a valve 46, an air trap 48, a flow meter 50, and out a pump core outlet 40. While FIG. 2 shows the filter 44, pump 36, valve 46, air trap 48, and flow meter 50 arranged along the flow path 11 in the order herein described, it is contemplated that these components may be arranged in a variety of other sequences along the flow path 11.

As shown in FIG. 2, the filter 44, pump 36, valve 46, air trap 48, and flow meter 50 are attached to a surface of a pump core base 52. In an alternative embodiment, the filter 44, pump 36, valve 46, air trap 48, and flow meter 50 are located within or partially within the pump core base 52. The fluid passes 42 are formed through or on a pump core base 52 and provide fluid communication between the components of the pump core 18. In certain embodiments, the pump core base 52 is formed of a layered structure of, for example, stainless steel such as SUS 304, or other similarly suitable rigid material. In certain embodiments, the fluid passes 42 are formed between the layers of material forming the pump core base 52.

With respect to the pump 36, it is contemplated that a variety of types of pumps, including peristaltic pumps, syringe pumps, and elastomeric pumps, can be employed as the pump 36. However, in order to achieve the greatest accuracy, compact size, and convenience, the pump core 18 is a microelectromechanical, or MEMS, micropump driven by a piezoelectric effect. In brief, small channels and chambers are formed in a multilayer structure, such as stainless steel, silicon wafer or other similarly rigid material. By attaching a thin piece of material, such as glass, on the surface of the layered structure, flow paths and fluid chambers are formed. A layer of piezoelectric material, or a piezoelectric body such as quartz, is attached to the glass on the side opposite the layered structure. When a voltage is applied to the piezoelectric body, a reverse piezoelectric effect, or vibration, is generated by the piezoelectric body and transmitted through the glass to the fluid in the chamber formed in the layered structure. In turn, a resonance is produced in the fluid in the chamber. Through the inclusions of valves, flow restrictions, and/or other design features in the fluid flow paths, a net directional flow of fluid through the chamber formed by the layered structure and the glass covering can be achieved. Examples of such pumps and related control systems are described in greater detail in the Assignee's copending U.S. patent application Ser. No. 12/972,348 entitled Infusion Pump and U.S. patent application Ser. No. 12/972,374 entitled Patient Fluid Management System, the contents of which are each herein incorporated in their entirety.

The filter 44 may be formed of, for example, a 20 micrometer stainless steel mesh and functions, in part, to prevent foreign particles from entering the pump 36 and flow meter 50. The valve 46 functions to prevent the free flow of fluid through the pump and thereby through the fluid flow path 11. The valve 46 may be formed of the same material or a different material as the pump core base 52 and may be formed separately or integrally with the pump core base 52. The valve 46 is configured, for example, to close or otherwise prevent flow of fluid when the pump 36 is not active or otherwise in operation. The air trap 48 is formed of a membrane filter such as, a Durapore membrane filter and is configured to trap bubbles of approximately 1 millimeter and larger.

The flow meter 50 may comprise a variety of known flow meters. For example, the flow meter 50 may be configured to determine fluid flow rates by employing a heater that heats the fluid being monitored and senses the flow of the heated fluid downstream of the heater. Such flow meters are available from Sensirion AG of Switzerland and Siargo Incorporated of the United States of America and are described in greater detail in at least U.S. Pat. No. 6,813,944 to Mayer et al. and U.S. Publication No. 2009/0164163, which are herein incorporated by reference. Alternatively, the flow meter 50 may be configured to employ two pressure sensors positioned on each side of a constriction within the fluid flow path 11. Fluid flow rates are determined by the relative difference between the pressure sensors and changes thereof. Alternatively, the flow meter 50 may function based on the principles of distortion. For example, flow rates may be determined by measuring the distortion of a membrane having an orifice that is interposed in a fluid flow path. In certain embodiments, compensation for temperature and viscosity for the fluid for which a flow rate is being determined will be performed with the assistance of databases and the controller 28.

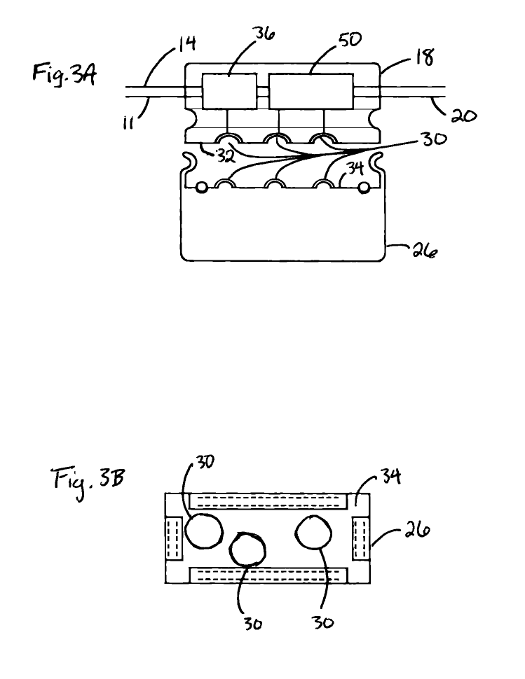

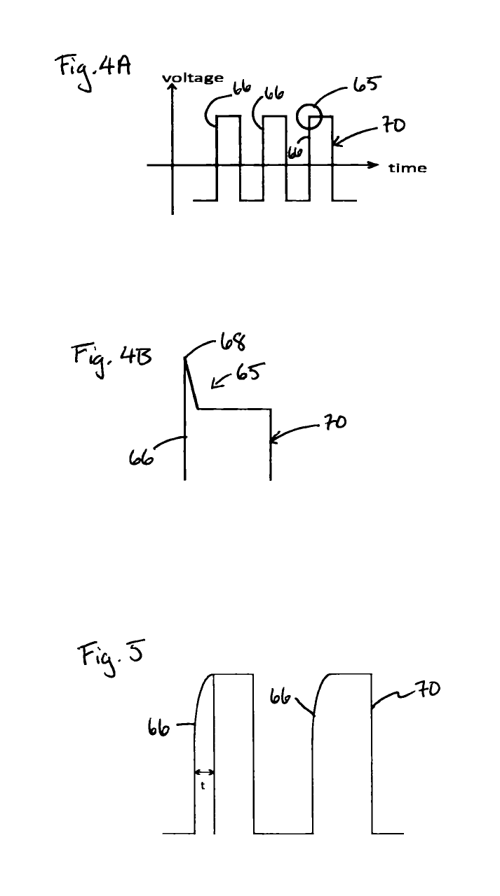

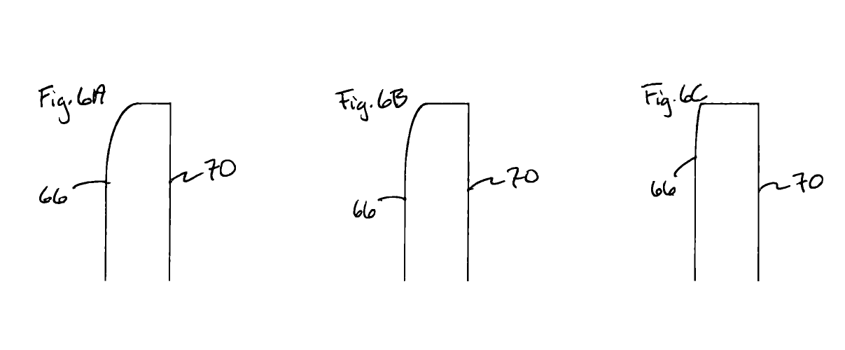

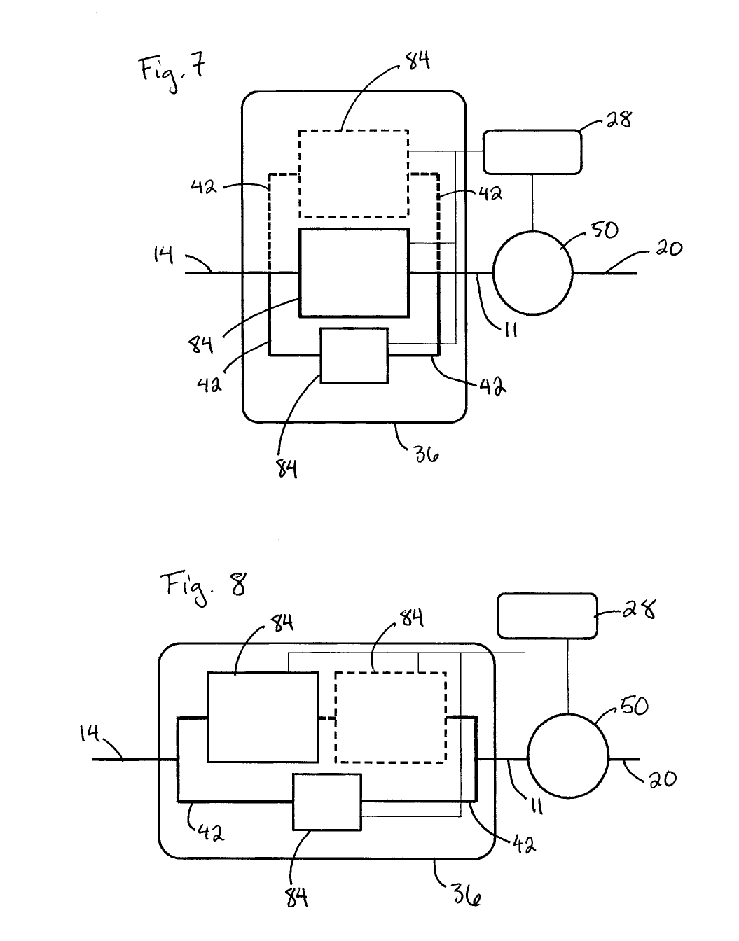

The pump stay 26 of the infusion system 10 houses the circuitry for providing power to the pump 36, for providing power to the flow meter 50, and for providing electrical communication of data from the flow meter 50 back to the controller 28. Hence, as shown in FIGS. 3A and 3B, in one embodiment of the present invention, the pump stay 26 employs a plurality of electrodes 30 for establishing electrical communication with the pump core 18. A first electrode 30 is associated with an electrical circuit configured to provide power with, for example 1 to 180 volts, to the pump 36 of the pump core 18 from the controller 28. A second and third electrode 30 are associated with an electrical circuit configured to provide power with, for example, a reference voltage of one to five volts to the flow meter 50 and to return a analogue or digital data signal from the flow meter 50 to the controller 28. In certain embodiments an amplifier is employed to amplify the data signal from the flow meter.

In certain embodiments of the present invention, the pump stay 26 incorporates memory and display features. In such a hybrid pump stay embodiment, the pump stay 26 need not be permanently networked or otherwise in continuous electrical communication with the controller 28. The pump stay 26 is operable to store and execute the infusion protocol. The hybrid pump stay is further operable to display certain information, for example, current operational data such as flow rates and system pressure, as well as data relating to the infusion protocol.

In operation, medical staff may carry a compact, mobile, control unit that employs an operator interface such as a touch screen or key pad. In order to program or prepare the hybrid pump stay 26 for execution of an infusion protocol, medical staff temporarily establishes electrical communication between the mobile controller and the hybrid pump stay 26 by, for example, connecting a wired coupling between the mobile controller and the hybrid pump stay 26 or by establishing wireless communication between the mobile controller and the hybrid pump stay 26. Medical staff may then manually enter or download a preconfigured infusion protocol to the hybrid pump stay 26, confirm the entry or download accuracy; start the infusion protocol, and then disconnect the mobile controller from the hybrid pump stay 26.

In this manner a hospital or other facility may utilize fewer control units to operate a greater number of infusion systems 10. Furthermore, in accordance with current trends in healthcare safety, while the hybrid pump stay allows for observation of certain real-time and infusion protocol data, the hybrid pump stay 26 does not allow for infusion protocol adjustment without the mobile controller being present. In other words, the hybrid pump stay 26 does not allow for the patient or other non-authorized person to adjust the infusion system 10 at the bed-side unless a mobile controller is also present.

As shown in FIGS. 3A and 3B, the pump core 18 and the pump stay 26 are formed such that the components can be physically attached to one another by employing elements such as recesses and deflectable binders that are complementary to one another. Such mating systems are described in further detail in the Assignee's copending U.S. patent application Ser. No. 12/972,348 entitled Infusion Pump and U.S. patent application Ser. No. 12/972,374 entitled Patient Fluid Management System, the contents of which are each herein incorporated in their entirety. Electrical communication is established between pump core 18 and the pump stay 26 through complementary electrodes 30 formed on a surface 32 of the pump core 18 and a surface 34 of the pump stay 26. In order that the pump core 18 and the pump stay 26 are mated in the proper orientation relative to one another, i.e. that the corresponding electrodes are properly mated to each other, the electrodes 30 on the pump core 18 are positioned in an asymmetric orientation that correspond to the asymmetric positioning of the electrodes 30 of the pump stay 26, as shown in FIG. 3B. In such a configuration, if the pump core 18 and the pump stay 26 are mated improperly, no electrical connection is established between the pump core 18 and the pump stay 26 and the infusion system 10 will be inoperable and/or provide the user with a notification or alert. In an alternative embodiment, asymmetric structural or visual features may be employed in the pump core 18 and the pump stay 26 such that it is obvious to a user that there is only one possible orientation for mating the pump core 18 and the pump stay 26. For example, the pump core 18 and the pump stay 26 may both be asymmetrically shaped or may employ correspondingly colored indicators making obvious the proper orientation of the components.

As shown in FIG. 1, in certain embodiments of the present invention, the controller 28 employs a power receiver 54 for receiving a universal 100-250 volt, alternating current. The current is, in turn, converted to, for example, a 2 to 7 volt, direct current by a power converter 56, such as those well known in the art for use in mobile personal computers. The controller 28 further employs a battery 58 for providing power to the infusion system 10 when power is not received through the power receiver 54, for example during transport of the system 10 while in use or during a power outage at a healthcare facility.

The controller 28 also employs a user interface 60 having a screen for user viewing and a user input portion for entering the desired infusion information and/or adjusting infusion parameters. The user interface 60 may be in the form of a touch operable screen and/or may employ data entry buttons or keyboards. For example the user interface 60 may be a liquid crystal touch panel display and may employ a reset or reboot button. Additionally, the controller 28 may employ one or more communications ports 64 in the form of local area network or universal serial, or other similar communication connection ports. Exemplary controllers 28 are further described in the Assignee's copending U.S. patent application Ser. No. 12/972,348 entitled Infusion Pump and U.S. patent application Ser. No. 12/972,374 entitled Patient Fluid Management System.

The controller 28 further employs a central processing unit, CPU, or other similar computing device operable to store and run software and/or firmware for operation of the infusion system 10. Broadly speaking such software may employ a first component configured to analyze a real-time or present infusion state or situation, and a second component configured to realize data inputs or instructions enter by medical staff through the user interface 60, determine needed adjustments, and provide the necessary signals to the system to realize the adjustments. In operation, a flow rate is input through the user interface 60 or is provided through the communication ports 64 of by medical staff. The software will break down or adopt the input flow rate relative to the specification of the infusion system 10 and then select the proper pump 36 or pumps 36 that match the demand. For example, in certain embodiments of the present invention, the software first recognizes the maximum potential flow rate of the infusion system 10. Then the software calculates if the demand is within the specifications of the system 10. If it is within the specification of the system 10, the software calculates which pump and/or fluid chambers will be activated and how the same will be operated in order to achieve such flow rate(s).

Once an infusion therapy is initiated, the software will monitor the information from flow meter 50 and calculate the amount of real-time fluid infused or accumulated fluid. If the ideal infusion schedule and the amount of real-time fluid infused or accumulated fluid is dissociated or not within a previously specified range of deviation, the software will calculates the new flow rate to required carry on the therapy and/or finish the therapy in order to achieve the ideal infusion schedule. For example, if (ideal infusion schedule)-(real-time infused or accumulated fluid) is negative, the flow rate is increased. If the difference is positive, the flow rate is decreased.

In certain embodiments of the infusion system 10 of the present invention, the infusion system 10 is operable to provide infusion flow rates that range of, for example, 0.1 to 1000 milliliters per hour. In order to provide such a relatively broad range of flow rates, some or all of the following parameters of the infusion system 10 are manipulated: (1) the frequency of the current provided to the pump 36; (2) the voltage of the current provided to the pump 36; (3) the manner in which the voltage is applied to the pump 36, i.e. the shape of the voltage curve applied to the pump 36; (4) the size and number of the pumps 36 or the size and number of the fluid chambers employed within a single pump 36; and (5) the back pressure applied downstream of the pump 36 in the fluid flow path 11. Generally speaking, the frequency of the current provided to the pump 36 is in the range of, for example, 0 to 300 Hertz or 0 to 200 Hertz, and the voltage provided to the pump 36 is in the range of, for example, 50 to 200 volts or 80 to 140 volts.

As shown in FIG. 4A, the shape of the voltage curve, i.e. the shape of the curve showing the voltage applied to the pump 36 relative to the time in which the voltage is applied to the pump 36 approximates a rectangular wave form 70. However, in certain circumstances when the voltage is applied as indicated in FIG. 4A, a leading edge 66 of the rectangular wave form 70 over shoots or progresses beyond the desired maximum voltage desired thereby resulting in a leading edge 55 having a voltage spike 68, as shown in FIG. 4B. FIG. 4B is an enlarge view of area 65 of the leading edge 66 shown in FIG. 4A. In certain circumstances, the voltage spike 68 may adversely affect the fluid flow rate and/or damage the pump 36. For example, the voltage spike 68 may cause a fracture or breakage of the piezoelectric body of the pump 36.

Hence, in order to address this potential problem, in certain embodiments of the present invention, a sloping, curved or otherwise softened leading edge 66 of the rectangular wave form 70 may be employed, as shown in FIGS. 5 and 6A-6C. In other words, the leading edge 66 is changed from a vertical line indicating an approximately single, instantaneous step up in voltage to an alternatively shaped line indicating a more gradual increase in voltage over a time “t”. The time t representing the time period from when voltage is initially increased to when the desired maximum voltage is achieved. The time t may, for example, range from 0.325 to 0.925 milliseconds, 0.425 to 0.825 milliseconds, or may be 0.625 milliseconds.

For example, if all other control parameters are maintained consistent and the rectangular wave form 70, shown in FIG. 6B, is considered as generating a reference flow rate, increasing the time t such as shown in FIG. 6A results in a relative decrease in the flow rate. Conversely, decreasing the time t such as shown in FIG. 6A results in a relative increase in the flow rate.

With respect to the size and number of the pumps 36, it is noted that the larger the pump 36, typically the lower the accuracy of the fluid flow rate of the pump 36. Accordingly, in order to achieve both relatively high and low flow rates from the infusion system 10, it may be desirable to employ multiple pumps 36 of varying sizes. In such a multi-pump 36 infusion system 10, each individual pump 36 will be associated with a separate piezoelectric body. Alternatively stated, each pump 36 is independently activated by the controller 28. As shown in FIG. 7, in certain embodiments of the present invention, the various pumps 36 are in fluid communication with one another in a parallel manner. Alternatively, as shown in FIG. 8 the infusion system 10 may be configured to locate the pump 36 having the same specification, for example the same size, shown as boxes of the same size in FIG. 8, in series and locate the pump 36 having different specifications in parallel.

An infusion system 10 according to the instant embodiment employ pumps 36a, 36b, 36c . . . 36n having different specifications, e.g. sizes. The system 10 may further employ n number of each of the pumps 36a, 36b, 36c . . . 36n. Each of the pumps 36a, 36b, 36c . . . 36n operable to achieve a maximum flow rate of max(36a), max(36b), max(36c) . . . max(36n), respectively. Accordingly, the maximum flow rate of the system 10 is calculated according to the formula:

Maximum Flow Rate=(Max(36a)(n))+(Max(36b)(n))+ . . . (Max(36n)(n))

The minimum flow rate for such an infusion system 10 would be the lowest possible flow rate achieved by activating only the smallest pump 36. For example, an infusion system composed of (1) two pumps 36 having maximum flow rates of 300 ml/h; and (2) two pumps 36 having maximum flow rates of 100 ml/h; and (3) two pumps 36 having maximum flow rates of 50 ml/h would be operable to generate flow rates ranging from a maximum flow rate of 1000 ml/h to the minimum flow rate of one of the 50 ml/h flow rate pump 36, for example 0.1 ml/h.

The pump 36 has a dimension, for example, a length and/or width, in the range of, for example, 4 to 18 millimeters; 7 to 15 millimeters; or 7 millimeters; or 15 millimeters.

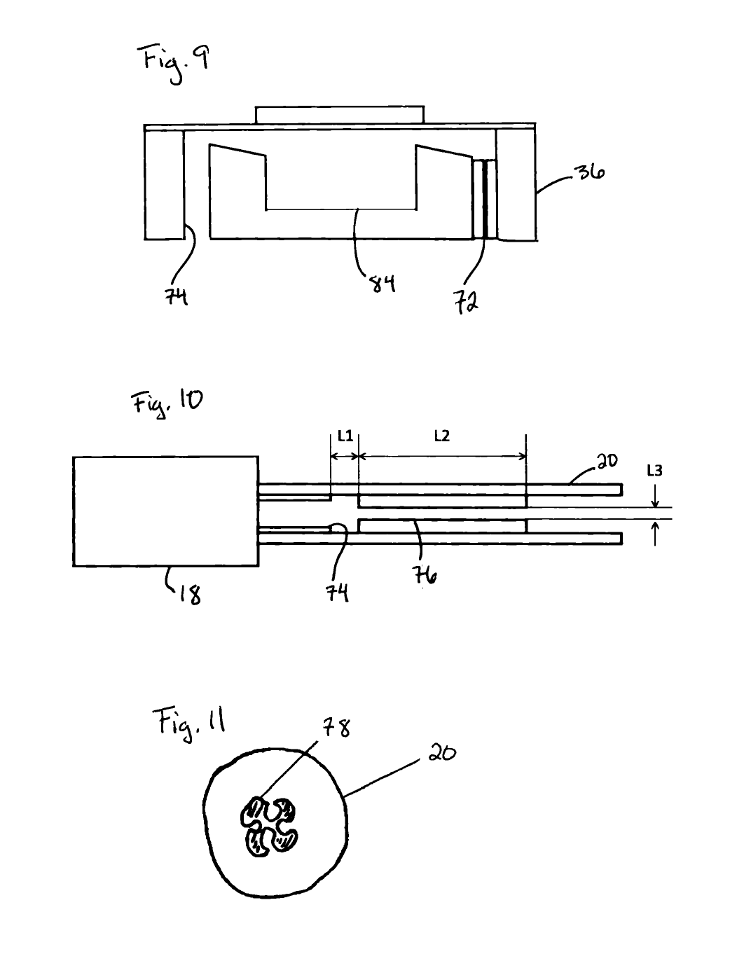

With respect to the control of the back pressure applied downstream of the fluid chamber(s) of the pump 36 in the fluid flow path 11, back pressure may be generated in one or a combination of various manners. Broadly speaking, the smaller the diameter of the fluid flow path 11 and the greater the length of the reduced diameter, the greater the resulting resistance and back pressure generated. For example, in certain embodiments of the present invention, as shown in FIG. 9, fluid flow resistance and thus back pressure is increased by forming a pump 36 with a narrow outlet channel 72 relative to the pump 36 inlet channel 74.

In another embodiment, shown in FIG. 10, the resistance is provided in all or a portion of the patient line 20. Wherein a distance L1 is representative of the distance from a rigid coupling 74 of the pump core 18 to the beginning of the reduced diameter portion 76 of the patient line 20. In embodiments employing an elastic patient line 20 formed of a material such as vinyl chloride, it is desirable to minimize the distance L1. A distance L2 is representative of the length of the reduced diameter portion 76, and a diameter L3 is representative of the diameter of the reduced diameter portion 76. The formula (L2/L3)² is representative of the relationship between a fluid flow rate and the distance L2 and the diameter L3.

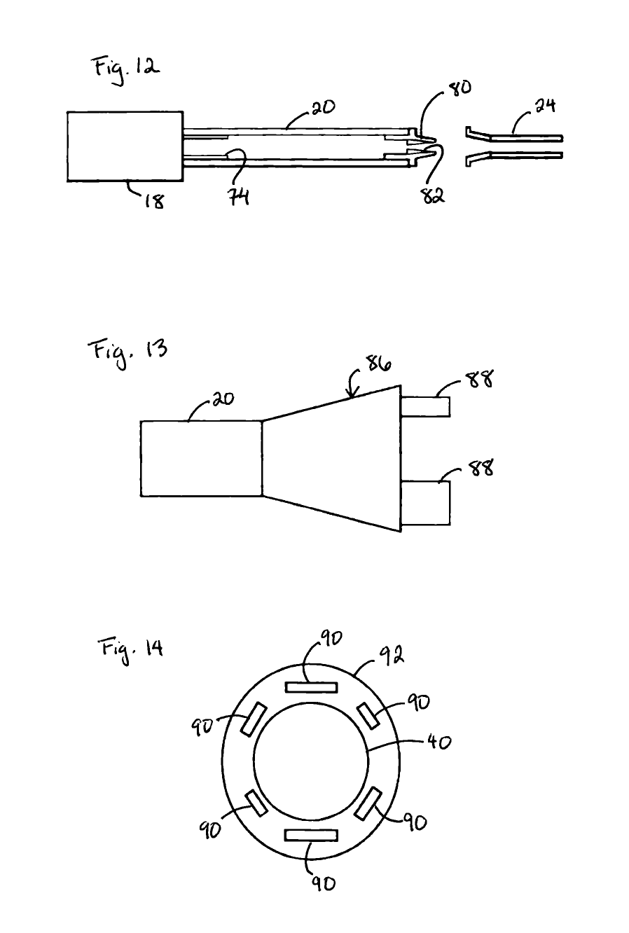

In yet another embodiment of the present invention, increased back pressure is achieved by increasing the surface area of the lumen of all or a portion of the patient line 20. For example, the patient line 20 may employ an irregular shaped lumen 78. Stated alternatively, tubing may be employed that has a lumen that is not circular in cross-section, as shown in FIG. 11. As the surface area of the lumen 78 increases relative to the volume of the lumen, the resistance and back pressure provided by the tubing increases.

In another embodiment of the present invention, increased back pressure is achieved through employing restrictive couplings within or at either end of the patient line 20. For example, as shown in FIG. 12, a restrictive coupling 80 having a lumen 82 reduced diameter or other restrictive feature is employed as the connector or interface between patient line 20 and the connector 24 leading into the patient's circulatory system22.

In view of the above-described flow control parameters, one embodiment of the present invention may achieve a minimum flow rate of, for example, 0.01-0.1 milliliters per hour, by employing, for example, the patient line 20 having an irregularly shaped lumen 82; a single 7 millimeter pump 36 to which approximately 80 volts is applied with a time t of approximately 0.825 and approximately 5-25 Hertz. A maximum flow rate of, for example, 100-1000 milliliters per hour, may be achieved by employing, for example, a standard patient line 20 not having an irregularly shaped lumen 82; a single 15 millimeter pump 36 to which approximately 140 volts is applied with a time t of approximately 0.425 and approximately 200 Hertz.

In view of the above-described embodiments in which the patient line 20 provides back pressure, it is further contemplated that a single infusion system 10 may be operable to function with different flow rate ranges by employing different restrictive patient lines 20 having different restrictive characteristics. Hence, in order to provide enhanced patient safety and ease of use, in certain embodiments of the present invention, infusion system 10 automatically recognizes and compensates for different restrictive or non-restrictive patient lines 20. For example, in operation, medical staff may set up an infusion system 10 by, in part, connecting an administrative set 14 to the inlet 38 of the pump core 18 and a patient line 20 to the outlet 40 of the pump core 18. According to one embodiment of the present invention, an interface between the outlet 40 of the pump core 18 and the patient line 20 allows for the infusion system 10 to identify the exact patient line 20 that is connected to the pump core 18 and to thereby use stored information regarding the specific patient line 20 that is connected in order to determine the implementation of the infusion protocol.

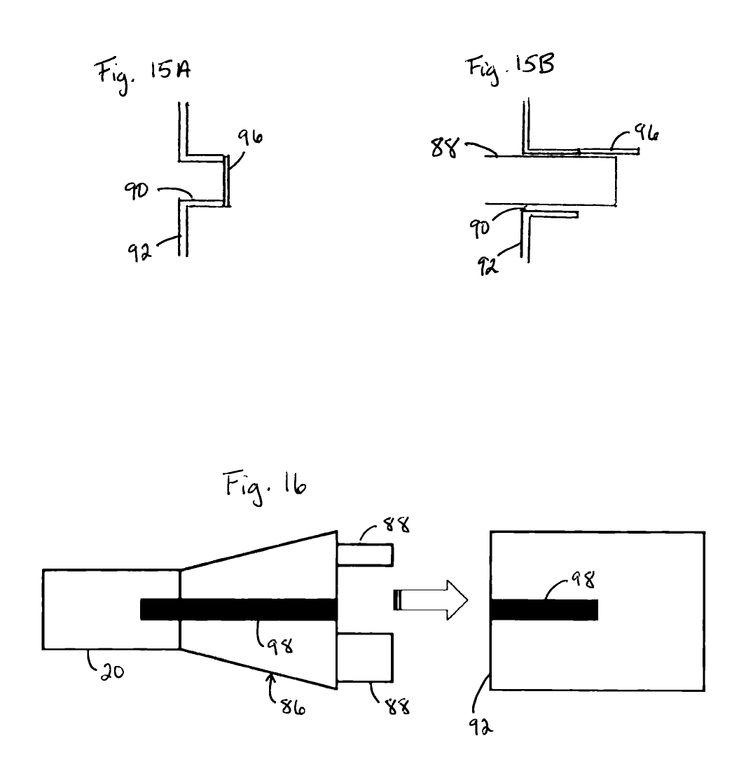

As shown below in FIG. 13, an end portion 86 of the patient line 20 employs one or more protrusions 88. The protrusions 88 are arranged so as to be complementary to receivers 90 employed in an outlet connector 92, shown in FIG. 14. The outlet connector 92 is attached to or incorporated into the pump core 18 and functions as one side of the interface between the patient line 20 and the pump core 18. The complementary side of the interface between the patient line 20 and the pump core 18 is the end portion 86 of the patient line 20. When the end portion 86 of the patient line 20 is connected to the outlet connector 92 of the pump core 18, the protrusions 88 are inserted into complementary receivers 90 located in the outlet connector 92 of the pump core 18. The protrusions 88 and receivers 90 are arranged so that the patient line 18 can be connected to the pump core 18 in only one rotational alignment. Once inserted into the receivers 90 of the pump core 18, the protrusions 88 actuate one or more switches.

In one embodiment, actuation of the switches by the protrusions 88 results in establishing, disrupting, or manipulating the resistance of one or more electrical circuits and thereby allows for a change in an electrical state of the one or more circuits. The specific change in electrical state of the circuit or circuits resulting from the connection of a specific patient line 20 is recognized by the controller 28 as being an indication that the specific patient line 20 is being employed in the infusion system 10.

In order for a single pump core 18 to receive and automatically recognize a variety of different patient lines 20, the outlet connector 92 employs receivers 90 that receive any of the combinations of protrusions that are present in the compatible patient lines 20. Alternatively stated, there may be more receivers 90 present on the outlet connector 92 than there are protrusions 88 present on any single patient line 20. The different patient lines 20 are distinguishable from one another by the different combinations; characteristics, such as length, width, and cross-sectional shape; and locations of the protrusions 88 employed on the end portion 86 of the patient line 20.

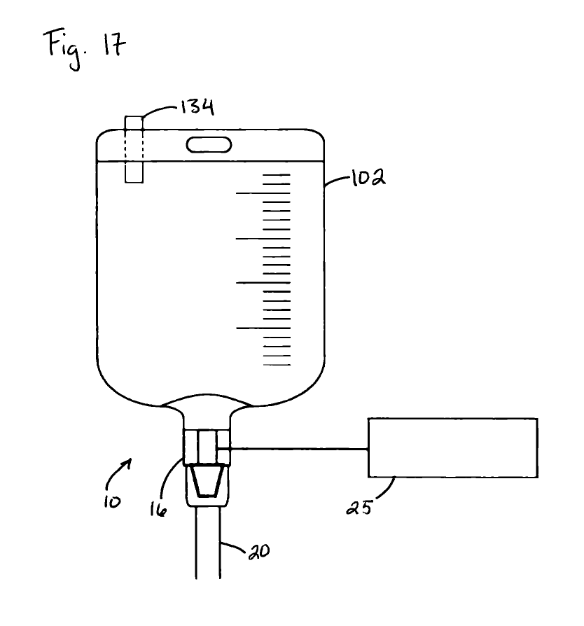

In one embodiment, the protrusions 88 activate dual in-line packaged, DIP, switches located within the receivers 90 of the outlet connector 92. In another embodiment, as shown below in FIGS. 15A and 15B, the switches are in the form of reversibly transposable elements 96 located within the receivers 90 of the outlet connector 92 that are displaced by insertion of the protrusion 88 into receivers 90.

In another embodiment, a portion of the protrusion 88, for example a tip of the protrusion 88 or one or more circumferences around the protrusion 88 are coated or otherwise made of a conductive material, such as metal. Insertion of the protrusion 88 into the receiver 90 functions to establish, disrupt, or manipulate the resistance of an electrical circuit, a portion of which is located within the outlet connector 92. In yet another embodiment of the present invention, upon insertion into the receivers 92, the protrusions 88 break or otherwise manipulate conductive elements, such as thin wires, that form an electrical circuit, a portion of which is located within the outlet connector 92. The breaking of the conductive elements establishes, disrupts, or manipulates the resistance of an electrical circuit, thereby providing a signal to controller 28 that allows the system to identify the specification of the attached tube set.

In order to assist the medical staff in connecting the patient line 20 and the pump core 18 which, in certain embodiments is operable to be connected in only one rotational orientation, one or more alignment elements 98 may be employed on the patient line 20 and the pump core 18. For example, as shown in FIG. 16, the alignment element 98 may be in the form of axial markings or coloration along a length of the patient line 20 and the outlet connector 92 and/or the pump core 18. Alternatively, the alignment element 98 may be a physical feature of the patient line 20 and the outlet connector 92, for example, the size and shape of one or more of the protrusions 88 and receivers 90 may function as an alignment element 98.

While the interface of the patient line 20 and pump core 18 has been described above as a longitudinally, insertion-based connection, in certain embodiments of the present invention, a threaded or rotational-based connection is employed alone or in combination with any of the above described features.

According to one embodiment of the present invention, as shown in FIG. 17, certain components of the infusion system 10, for example the pump core 18 or, alternatively, the pump core 18 and pump stay 26, are incorporated into a fluid bag 102. In certain embodiments, the fluid bag further incorporates an input port 134 for the augmentation of fluids into the interior of the bag 102. The input port 134 may be formed of, for example a non-inflectional injector, such as a sure plug or a clave connector.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A medical fluid infusion system comprising:
a fluid flow path comprising:
a pump;
a valve;
an air trap;
a flow meter;
a patient line; and
a fluid flow restriction independent of the valve.

2. The medical fluid infusion system of claim 1 wherein the fluid flow path further comprises fluid passes formed through a pump core base.

3. The medical fluid infusion system of claim 2 wherein the pump core base is formed of stainless steel.

4. The medical fluid infusion system of claim 1 further comprising a plurality of pumps.

5. The medical fluid infusion system of claim 1 further comprising a plurality of pumps having different dimensions.

6. The medical fluid infusion system of claim 1 wherein the fluid flow restriction forms a fluid outlet of the pump.

7. The medical fluid infusion system of claim 1 wherein the fluid flow restriction forms a portion of a lumen of the patient line.

8. The medical fluid infusion system of claim 1 wherein the fluid flow restriction forms a portion of a lumen of a connector that engages the patient line.

9. A fluid infusion system comprising:
a pump core having a fluid inlet and a fluid out let;
a pump stay reversibly attached to the pump core;
a patient line connected to an outlet of the pump core.

10. The fluid infusion system of claim 9 wherein the pump core and the pump stay comprise correspondingly asymmetric physical attachment features that prevent the attachment of the pump core to the pump stay except in a single orientation relative to one another.

11. The fluid infusion system of claim 9 wherein the patient line comprises an end portion having protrusions sized and shaped for insertion into receivers formed within the outlet of the pump core.

12. The fluid infusion system of claim 11 wherein the protrusions of the end portion of the patient line are sized and shaped to manipulate an electrical circuit within the outlet of the pump core.

13. The fluid infusion system of claim 12 further comprising a controller configured to determine an electrical state of the electrical circuit within the outlet of the pump core.

14. The fluid infusion system of claim 9 further comprising corresponding alignment elements located on the patient line and the outlet of the pump core.

15. A method for controlling a flow rate of an infusion system comprising the steps of:
receiving infusion flow rate instructions from a user interface;
recognizing an infusion pump configuration;
recognizing a fluid flow restriction configuration;
determining an infusion protocol based upon said steps of recognizing a infusion pump configuration and recognizing a fluid flow restriction configuration; and
advancing an electrical signal to the infusion pump according to the determined infusion protocol.

16. The method for controlling a flow rate of an infusion system according to claim 15 wherein the step of recognizing an infusion pump configuration comprises recognizing a quantity or size of a plurality of pumps.

17. The method for controlling a flow rate of an infusion system according to claim 15 wherein the step of recognizing a fluid flow restriction configuration comprises recognizing automatically a portion of an infusion tube set attached to the infusion pump.

18. The method for controlling a flow rate of an infusion system according to claim 15 wherein the step of advancing an electrical signal to the infusion pump according to the determined infusion protocol comprises providing a voltage to the infusion pump that increases from a minimum to a maximum over a time in the range of 0.325 to 0.925 milliseconds.

19. The method for controlling a flow rate of an infusion system according to claim 15 wherein the step of advancing an electrical signal to the infusion pump according to the determined infusion protocol comprises providing 50 to 200 volts to the infusion pump.

20. The method for controlling a flow rate of an infusion system according to claim 15 wherein the step of advancing an electrical signal to the infusion pump according to the determined infusion protocol comprises providing a electrical signal having a frequency of 0 to 300 Hertz to the infusion pump.
